(19) United States
(12) Patent Application Publication
(10) Pub. No.: US 2001/0024656 A1
(43) Pub. Date: Sep. 27, 2001

(54) Novel wound dressing, process of manufacture and useful articles thereof

(76) Inventor: Mark E. Dillon, Menungie, PA (US)

Correspondence Address: John F. A. Earley III, 86 The Commons At Valley Forge East, 1288 Valley Forge Road, P.O. Box 750, Valley Forge, PA, 19482-0750, US

(21) Appl. No.: 09/754,010

(22) Filed: Jan. 3, 2001

Related U.S. Application Data
(60) Provisional application No. 60/174,477, filed on Jan. 3, 2000.

(51) Int. Cl. A61F 13/00; A61K 9/70

(52) U.S. Cl. 424/443

(57) ABSTRACT

A wound dressing is provided that serves a unique dual-purpose role. One surface of the dressing is comprised of a polyurethane foam and the other surface is comprised of a non-adherent thin-film of polydimethylsiloxane and polytetrafluoroethylene interpenetrating polymer networks (“IPN”). With the foam side of the dressing down against the wound, the product provides an adhesive surface for difficult fixation conditions. With the IPN side of the dressing against the wound, the dressing provides a non-adherent covering for fragile and sensitive wounds.

### BACKGROUND OF THE INVENTION

1. Field of the Invention

This invention relates to a novel wound dressing design. Particularly, this invention relates to a wound dressing which incorporates two distinct layers, each providing useful features and together providing a novel method of managing a variety of wound types. Ease of use, patient comfort and the cost of care are improved.

2. Description of the Prior Art

In the field of woundcare there exist several general categories of commonly used dressings. Some dressings aggressively adhere to the wound surface. For example, conventional gauze integrates into the wound as healing occurs and eschar forms on the wound surface. Other types of dressings are designed to adhere to the surrounding intact tissue around the wound site, but not directly to the wound. Examples of this type of dressing include polyurethane films coated with pressure sensitive adhesive. Other types of dressings are designed to be substantially nonadherent. Examples of this type include polyethylene oxide hydrogels, and particularly the material described in U.S. Pat. No. 4,832,009. The latter example is a dressing made from an interpenetrating polymer network (“IPN”) of polytetrafluoroethylene and silicone, and is presently marketed by Bio Med Sciences, Inc. of Allentown, Pa. as Silon-TSR® Temporary Skin Replacement. Each type of dressing has its advantages and disadvantages, and is indicated for certain wound conditions and user preferences.

There are a wide variety of wound types. Wounds can be categorized as chronic or acute. Examples of chronic wounds include venous stasis ulcers, decubitus ulcers and diabetic ulcers. Examples of acute wounds include burns, skin graft donor sites, skin graft recipient sites, abrasions and the like. The features required for the proper performance of a wound dressing depend on the wound type as well as the location of the wound on the body. For example, non-adherent films minimize disruption of fragile skin during dressing changes, but are not always applicable because of difficulties in keeping the dressing in position. This is particularly a challenge for skin graft donor sites on the back or buttocks of a patient, where ordinary movement and contact with bedding can easily dislodge the dressing. As a result, adhesive dressings are typically used for this type of wound. An additional example includes the use of absorbent dressings on chronic wounds. Chronic wounds tend to produce copious amounts of exudate which makes the use of thin film dressings difficult since these dressings are generally poor at managing wound fluid.

Even the same wound may require different dressings at different stages of the healing process. A venous stasis ulcer will produce copious amounts of exudate in the early stages of healing. Hydrocolloid dressings are often used on these wounds because of their high absorption capabilities. But as a wound of this type heals, the fragile epithelium can easily be damaged during dressing changes, so a non-adherent dressing may be substituted later in the healing process even if it is not as absorbent.

Bio Med Sciences, Inc. manufactures a thin-film non-adherent dressing made from an interpenetrating polymer network (“IPN”) of polytetrafluoroethylene and silicone (Silon-TSR®). The IPN film is flexible and thin (50 microns), thereby providing transparency and good conformity to wound contours. Small fenestrations are cut through the film so that wound fluid can wick away from the wound surface and be collected in a secondary dressing such as gauze. The outer gauze may be changed as required, but the IPN dressing may be left in place until the wound heals or for up to 10 days.

The IPN dressing is well-suited for applications such as laser resurfacing, which is a cosmetic surgery procedure almost exclusively performed on the face. The product's non-adherent and transparent properties provide clinical advantages during the healing process. This product, however, does not perform as well on certain other types of wounds, such as skin graft donor sites and many types of chronic wounds. The non-adherent character of the product is problematic for application on any part of the body where shear forces, such as contact with bedding or other surfaces, may cause the dressing to roll-up or slide off of the wound. This difficulty is particularly acute on lower limbs where the general shape tends to be somewhat conical thereby causing the dressing to slide distally.

The IPN dressing manufactured by Bio Med Sciences, Inc. provides desirable properties with respect to a conformable non-adherent surface for wound coverage. These features, however, have proved to be problematic with respect to maintaining wound coverage and avoiding dressing roll-up and slippage.

### SUMMARY OF THE INVENTION

In an effort to mitigate said problematic characteristics, I have unexpectedly created a dressing with a unique dual-purpose design.

The new dressing comprises a thin layer (50 microns) of the IPN material laminated to a polyurethane foam of approximately 1,500 microns in thickness. This construction has the effect of providing a greater cross-sectional thickness, which tends to be more resistant to roll-up, wrinkling and slippage.

By applying the dressing to the wound site with the IPN surface against the wound surface, the nonadherent advantages of the IPN material are preserved. At the same time, however, the foam layer minimizes any tendency for the dressing to slip, roll-up or wrinkle. Fenestrations are still cut through the IPN material and the foam passes wound exudate through to a secondary dressing.

Unexpectedly, I have discovered that the dressing of this invention is also useful for woundcare when used “up-side-down” with the foam layer against the wound instead of the IPN layer. This serves to provide a dressing with a higher level of surface adhesion but otherwise similar features. Wound fluid is still wicked from the wound surface to a secondary dressing and slippage or roll-up are still minimized.

This invention provides a single dressing that can offer disparate wound healing features depending on its orientation on the wound surface. This is useful for broadening the range of clinical applications for which either the IPN material or the foam layer could be used individually. This is true for different clinical cases or for the same case at different stages of the healing process.

While the two opposite approaches to wound healing (adhesive/non-adhesive) are commonly found in the field, no product combines these two features in a single dressing by means of simply using it one side up or the other. This provides great utility in the field where the number of products stocked is always minimized to reduce inventory costs. In addition, cost effectiveness is promoted due to consolidated manufacturing and distribution operations. Most importantly, this invention provides a unique dual-purpose dressing for a wide variety of wound types.

### BRIEF DESCRIPTION OF THE DRAWINGS

FIG. 1 shows a cross-sectional view of a preferred embodiment of this invention. The IPN material 10 is bonded to a foam layer 20 by means of silicone elastomer 30.

FIG. 2 shows a plan view of a dressing 40 cut from the material of this invention. Fenestrations 50 are cut through the IPN film to provide a means for managing wound exudate.

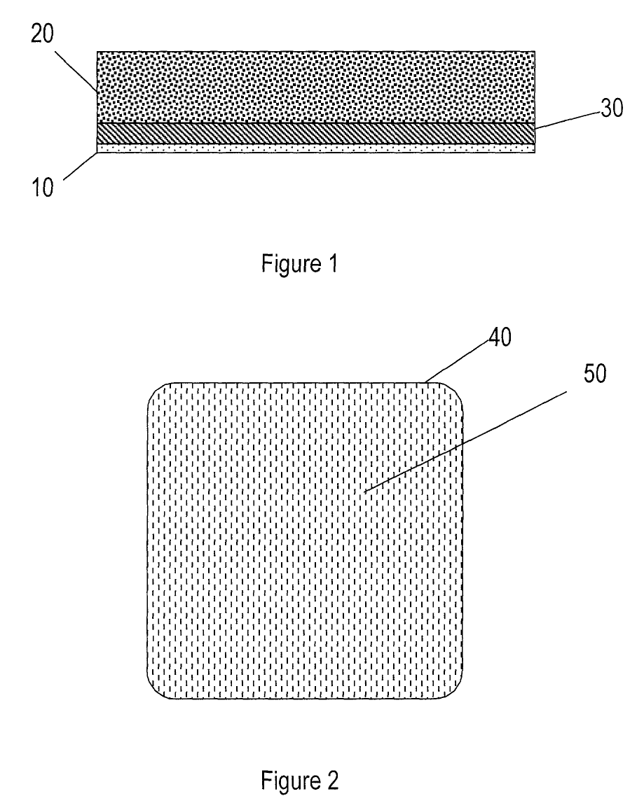

### DETAILED DESCRIPTION OF PREFERRED EMBODIMENTS

Turning to the drawings, there is shown the inventive new dressing which comprises a thin layer (50 microns) of IPN material 10 laminated to a polyurethane foam 20 of approximately 1,500 microns in thickness. Preferably, a silicone elastomer 30 is used to bond the IPN material 10 to the polyurethane foam 20. Fenestrations 50 are cut through the IPN film to provide a means for managing wound exudate.

The following examples are not intended to be limiting, as minor variations on these designs and processes would be obvious to those skilled in the art. Likewise, it is believed that other materials could be used to achieve the same dressing design.

### EXAMPLE 1

A continuous sheet of polydimethylsiloxane and polytetrafluoroethylene IPN was manufactured according to established methods. The film measured approximately 50 microns in thickness. The IPN film was then passed through a knife-over-roll assembly and coated with approximately 200 microns of liquid silicone rubber MDX4-4210 from Dow Corning Corporation of Midland, Mich. Soon after the silicone rubber was applied to the IPN material, an open-cell hydrophilic foam (Amrel® Medical Foam from Rynel Limited, Inc. of Boothbay, Me.) was laid onto the uncured silicone rubber and the laminate was passed through a tunnel style oven at approximately 150° C. for approximately 6 minutes. The resultant material was then fed through a rotary die-cutting apparatus to cut individual dressings from the sheet and to create fenestrations in the IPN film.

### EXAMPLE 2

The process of Example 1 was repeated with a pigment added to the liquid silicone rubber prior to the lamination process. A blue silicone-based ink (product code R1008-7 from Nusil Technology of Carpinteria, Calif.) was mixed into the MDX4-4210 at a concentration of 4 percent by weight. Since the IPN material is transparent and the foam is opaque, the blue pigment imparted a soft blue coloration to one side of the dressing. This serves as a visual indicator for differentiating one side of the dressing from the other in the field.

1. A wound dressing providing disparate wound management features depending on its orientation on the wound surface.

2. The product of claim 1 wherein one orientation provides a non-adherent surface and the other provides an adherent surface.

3. The product of claim 1 wherein the wound dressing consists of a multilayered composite structure.

4. The product of claim 2 wherein the wound dressing consists of a multilayered composite structure.

5. The product of claim 3 wherein the multilayered composite consists of at least one membrane layer and one foam layer.

6. The product of claim 4 wherein the multilayered composite consists of at least one membrane layer and one foam layer.

7. The product of claim 5 wherein the membrane layer consists of a silicone-containing compound.

8. The product of claim 6 wherein the membrane layer consists of a silicone-containing compound.

9. The product of claim 5 wherein the foam layer consists of a polyurethane material.

10. The product of claim 6 wherein the foam layer consists of a polyurethane material.

11. The product of claim 5 wherein the membrane layer consists of a silicone-containing compound and the foam layer consists of a polyurethane material.

12. The product of claim 6 wherein the membrane layer consists of a silicone-containing compound and the foam layer consists of a polyurethane material.

13. A method of manufacturing a wound dressing with disparate wound management features depending on its orientation on the wound surface consisting of (1) producing a thin film of a polymer compound, (2) passing said thin film through a coating assembly and depositing a layer of an adhesive substance, (3) causing a foam material to make intimate contact with said adhesive, and (4) optionally cutting individual dressings from the sheet and/or creating fenestrations in the film.

14. A method of managing a variety of wound types using a dressing with disparate wound management features depending on its orientation on the wound surface.

15. A new wound dressing, substantially as herein described.

16. A new method of manufacturing a dual-purpose wound dressing, substantially as herein described.

17. A new method of managing a variety of wound types, substantially as herein described.

18. A composite article for use as a wound dressing, said article comprising:
a first surface; and
a second surface;
wherein said first surface and said second surface each has disparate properties for wound healing.

19. A composite article for use as a wound dressing, said article comprising:
at least one first layer of material; and
at least one second layer of material bonded with said first layer of material;
wherein said at least one first layer of material forms a first surface of said article;
wherein said at least one second layer of material forms a second surface of said article; and
wherein said first surface and said second surface each has disparate properties for wound healing.

20. The composite article of claim 19, further comprising a bonding agent for bonding said at least one first layer of material to said at least one second layer of material.

21. The composite article of claim 20, wherein said bonding agent comprises a silicone compound.

22. The composite article of claim 19, wherein said at least one first layer comprises at least one membrane layer.

23. The composite article of claim 19, wherein said at least one first layer comprises a silicone-containing compound.

24. The composite article of claim 19, wherein said at least one second layer comprises a foam layer.

25. The composite article of claim 24, wherein said foam layer comprises polyurethane.

26. The composite article of claim 23, wherein said silicone-containing compound comprises an interpenetrating polymer network of polytetrafluoroethylene and silicone.

27. The composite article of claim 24, wherein said silicone-containing compound comprises an interpenetrating polymer network of polytetrafluoroethylene and silicone.

28. The composite article of claim 23, wherein said foam layer comprises polyurethane.

29. The composite article of claim 24, wherein said foam layer comprises polyurethane.

30. The composite article of claim 19, further comprising a pigment associated with at least one of said at least one first layer and said at least one second layer for distinguishing at least one of said at least one first layer and said at least one second layer from the other.

31. The composite article of claim 19, wherein said at least one first layer comprises a substantially transparent material, and wherein and said at least one second layer comprises a substantially opaque material, said composite article further comprising a pigment for imparting a discernable color to said at least one first layer of material.

32. The composite article of claim 20, further comprising a pigment; wherein said bonding agent comprises a silicone-containing compound, and wherein said pigment is added to said silicone containing compound.

33. The composite article of claim 19, wherein said at least one first layer comprises a polymer film with fenestrations therein.

34. A method for treating a wound comprising the steps of:
providing a wound dressing having at least a first treatment property when oriented in a first position and at least a second treatment property when oriented in a second position;
selecting one or the other of the first treatment property and the second treatment property for the wound being treated;
applying the wound dressing to a wound by orienting the wound dressing to contact the wound to deliver the selected treatment property.

35. The method of claim 34, wherein the step of providing a wound dressing having at least a first treatment property when oriented in a first position and at least a second treatment property when oriented in a second position comprises providing a wound dressing having at least a first surface and at least a said second surface;
wherein the step of selecting one or the other of the first treatment property and the second treatment property for the wound being treated comprises selecting one or the other of said first surface of said wound dressing and said second surface of said wound dressing; and
wherein the step of applying the wound dressing comprises contacting said wound with said selected surface.

36. The method of claim 34, further comprising the steps of:
allowing said wound dressing to remain in contact with said wound; and
removing said wound dressing from said wound.

37. The method of claim 34, wherein the step of providing a wound dressing comprises providing a wound dressing having at least one first layer and at least one second layer, wherein each layer has disparate properties for wound healing; wherein the step of selecting includes selecting one or the other of the first treatment property and the second treatment property for the wound being treated; and and wherein the step of orienting comprises positioning one or the other of said at least one first layer and said at least one second layer to contact the wound to be treated to apply the selected treatment property.
